(19) United States
(12) Patent Application Publication
(10) Pub. No.: US 2004/0067488 A1
(43) Pub. Date: Apr. 8, 2004

(54) Method and system for accepting order of manufacturing dna chip

(76) Inventor: Yuichi Takegawa, Hatogaya (JP)

Correspondence Address: Crowell & Moring, Evenson Mckeown Edwards & Leneha, Intellectual Property Law Group, 1001 Pennsylvania Avenue N W, Washington, DC, 20004-2595, US

(21) Appl. No.: 09/980,126

(22) PCT Filed: Jan. 31, 2001
(86) PCT No.: PCT/JP01/00683

(51) Int. Cl. G06F 17/60; C12Q 1/68; C12M 1/34

(52) U.S. Cl. 435/6; 435/287.2; 705/2

(57) ABSTRACT

To allow an order-placing party to prepare an experiment schedule efficiently by receiving an order for a DNA chip (microarray), prepare a production plan efficiently, and to inform the order-placing party of an experiment protocol, the delivery date, and the price.

A DNA chip order placing system receives an order for a DNA chip from a customer at any time. An order receiving system stores an inventory, a production plan, a method for calculating price, and a method for scheduling production, creates an optimal experiment protocol based on this information and information on the order received from the customer, prepares a production schedule, determines the delivery date, calculates the price, and notify the customer of them promptly.

An order-receiving party receives the order for the DNA chip, and prepares the production plan efficiently. The customer (order-placing party) promptly knows the experiment protocol, the delivery date, and the price, thereby preparing an experiment schedule efficiently.

### FIELD OF THE INVENTION

The present invention relates to a device [for] capable of receiving orders [for] that relate to producing DNA microarray chips [(microarrays),] and [for] analyzing genes [by] using the DNA chips.

### BACKGROUND ART BACKGROUND OF THE INVENTION

Japanese application patent laid-open publication No. 2000-279169 describes a method and a device for separating target polynucleotides. This publication specifically describes the method for separating polynucleotides comprising steps of immobilizing single-stranded oligonucleotide probes including a specific base sequence individually in multiple areas on a surface of a substrate, supplying specimen solution including polynucleotides on the substrate, bonding individual polynucleotides having complementarity with the individual probes by cooling the specimen solution after heating it to a predetermined temperature, replacing the solution on the substrate from the specimen solution with solution not including the polynucleotides, and increasing temperature of the surface of the substrate in one area of the multiple separate areas on the substrate to dissociate and recover only the polynucleotides [complementally] complementarily connected with the probe immobilized to the area.

Japanese application patent laid-open publication No. 2000-295990 describes a method for immobilizing DNA pieces where solution produced by [solving] dissolving or diffusing DNA [pieces] fragments and hydrophilic polymer in a [water-base] water-based medium is attached as a point on a surface of a solid phase carrier to bond the DNA [pieces] fragments on the surface of the carrier.

Japanese application patent laid-open publication No. 2000-210082 describes a method for immobilizing target DNA.

### INVENTION DISCLOSURE SUMMARY OF THE INVENTION

A purpose of the present invention is to provide a method and a system for accepting orders of [commission] commissioned production of DNA chips, [when] which allows for the efficient and quick preparation of a production plan.

[Also a] Another purpose of the present invention is to provide a method of accepting orders of DNA chip production, efficiently making a production plan [efficiently], and informing a requester of experiment protocol, price and delivery date quickly so that the requester can effectively make their experiment schedule [effectively].

The experiment [here] includes an inspection and a test using DNA chips.

The order receiving system for DNA chips of the present invention is provided with a memory device [storing] to store the content of at least one order[,] and the production cost and [the] control conditions for at least one type of DNA chip, such as information relating to experiment conditions including at least a temperature condition, and a processing device for calculating prices of individual orders by referring to the content of the orders and the production prices for the DNA chips.

[This] In the memory device, the system may [also] store information on production management and inventory in addition to the production prices of the DNA chips [in the memory device], and may calculate the delivery date while considering the information.

This system receives the contents of [received] orders for the DNA chips from customers at any time, and calculates prices of the individual orders by comparing the contents of the orders with the cost information. Also, the system calculates delivery dates by comparing the contents of the orders with an inventory status and a production schedule. With these calculations, a customer can immediately make a decision on the order[,] and prepare a plan for experiments. [Also the] The customer can also immediately prepare a [plan for] production plan[,] and start the production at a proper time.

Preferably the processing system communicates with a production control system and an inventory control system at any time or [periodically] on a periodic basis to automatically update production control information and inventory control information, respectively. This allows [calculating] price calculation for an order and also a delivery date based on the latest production control information and [the] inventory information[,].

The system can also store production prices and methods for optimizing [experiment] experimental conditions for many types of DNA chips in the memory device, and can [calculates] calculate delivery dates in addition to prices required for production and analysis.

As an output method for the prices and the delivery dates, a browser immediately notifies customers of [them] these items through the Internet.

As an example of the output form for the applied experiment conditions, a method for displaying optimized positions of nucleic acid probes mapped on a screen for indicating a DNA chip can be adopted. With this form, a customer can easily know the arrangement of the nucleic acid probes.

A customer can know the status of a received order in addition to the price and the delivery date at any time.

When a customer asks for only [production by commission] the custom-made DNA chips, and conducts an experiment by [himself] themself, the customer can install an optimized experiment protocol through media such as different types of storages, semiconductor memories, and communication lines.

The system of the present invention is typically realized by a computer on the Internet, and programs including plug-ins for that purpose are installed through media such as different types of storages, semiconductor memories, and communication lines.

### BRIEF DESCRIPTION OF THE DRAWINGS

FIG. 1 is [an overview schematic] a schematic overview of an order receiving system for DNA chips[.];

FIG. 2 is a flowchart for placing and receiving an order[.];

FIG. 3 is a screen shot for selecting probe type[.];

FIG. 4 is a screen shot for entering probe information [on probes.];

FIG. 5 is a screen shot for confirming entered probe information on probes[.];

FIG. 6 is a screen shot for selecting an analysis by commission[.];

FIG. 7 is a screen shot for entering a sample shipment date [of shipping a sample.];

FIG. 8 is first a screen for confirming an experiment protocol [(1).];

FIG. 9 is second a screen for confirming an experiment protocol[(2).];

FIG. 10 is a third screen for confirming an experiment protocol[(3).];

FIG. 11 is a screen for downloading an experiment protocol[.];

FIG. 12 is a screen for confirming an order content[.];

FIG. 13 is a screen for [showing] displaying price and delivery date[.];

FIG. 14 is a screen for showing completion[.];

FIG. 15 [is a figure showing a] shows the constitution of a system for receiving orders for DNA chips[.];

FIG. 16 [is a figure showing a] shows the content of a production schedule information file[.];

FIG. 17 [is a figure showing a] shows the content of a DNA chip information file[.];

FIG. 18 is a display screen for an experiment protocol[.];

FIG. 19 is a display [screed] screen for an experiment protocol[.];

FIG. 20 [is a figure showing a] shows the content of an inventory information file[.];

FIG. 21 is a figure showing a content of an order information file.

FIG. 22 is a figure showing a content of a probe arrangement information file.

FIG. 23 is a figure showing a content of an optimization information file.

FIG. 24 is a figure showing a content of an image information file.

FIG. 25 is a flowchart for registering production schedule information.

FIG. 26 is a flowchart for registering inventory information.

FIG. 27 is a flowchart for registering chip information.

FIG. 28 is a flowchart for registering optimization information.

FIG. 29 is a flowchart for registering order information.

FIG. 30 is a flowchart for referring to an experiment protocol.

FIG. 31 is a figure indicating a screen showing a probe arrangement.

FIG. 32 is a figure indicating a screen showing experiment control conditions.

FIG. 33 is a figure indicating a screen showing a DNA chip.

FIG. 34 is a figure indicating a screen showing an experiment protocol.

FIG. 35 is a flowchart for referring to a price.

FIG. 36 is a flowchart for referring to a delivery date.

### DESCRIPTION OF THE BEST EMBODIMENT

The following describes embodiments of the present invention based on figures.

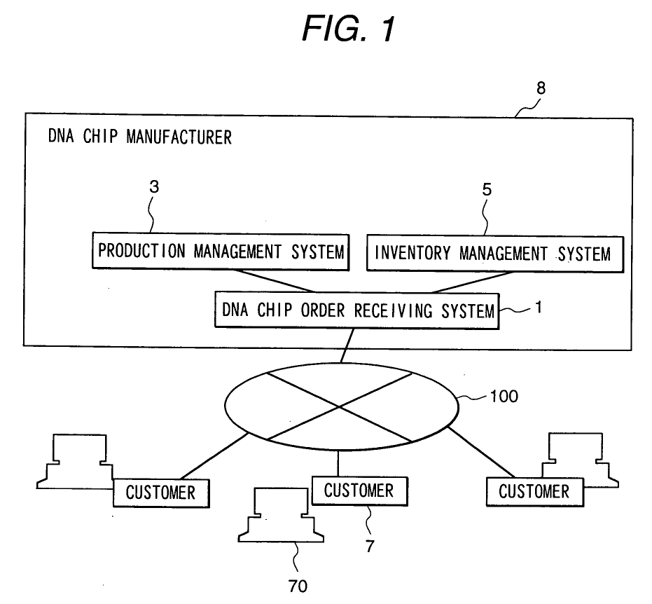

FIG. 1 shows an overall system constitution for an order receiving system for DNA chips relating to one embodiment of the present invention for realizing an order receiving system described below. In this figure, an order placing system 70 for a customer 7 is connected with a DNA chip order receiving system 1 of a DNA chip manufacturer 8 through the Internet 100. The DNA chip order receiving system 1 is connected with a production control system 3, an inventory control system 6, and other systems owned by the DNA chip manufacturer 8. With this constitution, an order receiving method and a manufacture meeting needs of customers are realized as described later.

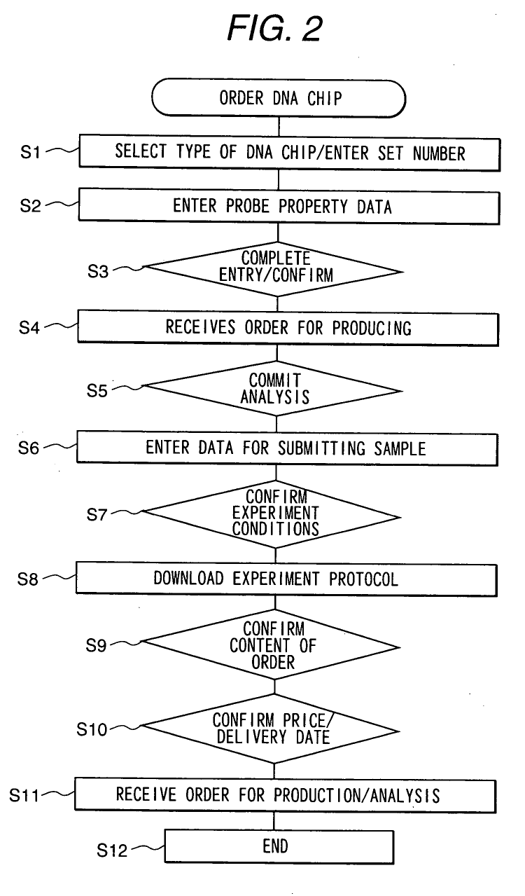

FIG. 2 is a flowchart for indicating order receiving steps accompanying placing an order.

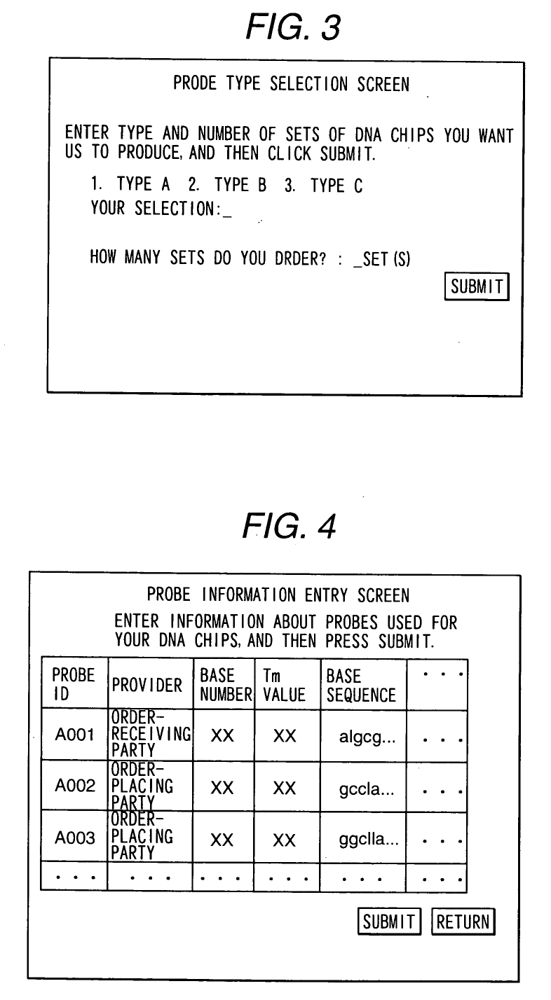

A customer (party asking for [commission] commissioned DNA chips) places an order for DNA chips. The customer uses an input device of the order placing system 70 to select the type of a DNA chip to be ordered, and enters the type and the set number through a communication network on the Internet 100 (S1). A screen shown in FIG. 3 is a screen used as an entrance (screen for selecting probe type), and the type is selected from Type A, Type B and Type C in the present embodiment. The Type A, the Type B, and the Type C mean a type for a company A, a type for a company B, and a type for a company C, for example. It is not necessary to limit the type selection to three types. Also a question, “How many sets do you order?”, is shown to display and enter the number of sets[; then]. Once the customer enters the information, they press [an] the enter button. Then, property data for the probes are entered (S2).

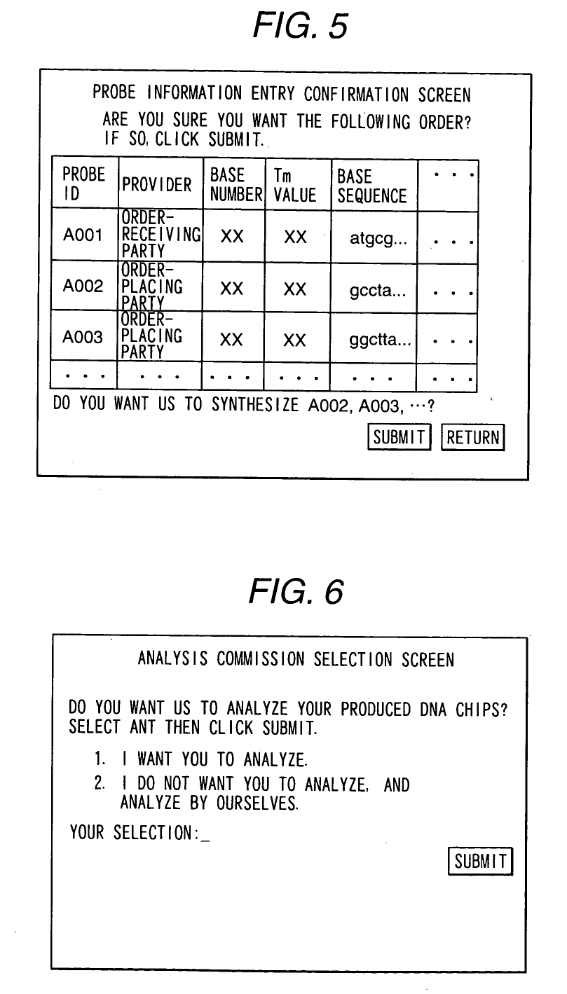

A screen shown in FIG. 4 is a screen used for the entry (Probe Information Entry Screen). Information on the probes (nucleic acid probes) used for the DNA chip, such as Probe ID, Provider, the number of bases, Tm (melting temperature) value, Base sequence, and other information, is entered, and Enter is clicked. The entered information is confirmed (Complete entry/confirm) (S3). The screen shown in FIG. 5 is a screen used for this step (Probe Information Entry Confirmation Screen). The input is confirmed to be correct on this screen, the arrangement of the probes is set, a screen for indicating the probe property data comprising the probes and the probe arrangement is shown on a screen, and the production order is received (S4).

This provides an order receiving method for producing DNA chips by immobilizing nucleic acid probes including a specific base sequence in individual independent areas on a surface of a substrate. Specifically, this is a method for receiving orders for producing DNA chips comprising steps of receiving types and the number of sets of DNA chips through a communication network, setting an arrangement of nucleic acid probes in the individual areas, displaying a figure indicating the nucleic acid probes and the property data of the probe positions of the individual nucleic acid probes on screen, sending the display screen through the communication network to ask for confirmation, and receiving the order-for production based on the display screen confirmed through the communication network.

Also, a method for receiving an order for producing DNA chips comprising steps of receiving types and the number of sets of the DNA chips through the communication network, setting an arrangement of the nucleic acid probes in the individual areas, displaying a figure indicating the nucleic acid probes and the control conditions of individual probe positions of the individual nucleic acid probes on screen, sending the display screen through the communication network to ask for confirmation, and receiving the order for production and analysis based on the display screen confirmed through the communication network is provided.

In this case, it may be possible to provide a price calculated by referring to price information and the like from a memory device for storing cost information on producing DNA chips.

Then, it is determined if an analysis is committed (S5).

The screen shown in FIG. 6 is a screen presented in this processing (Analysis Commission Selection Screen). The screen asks if an analysis using the produced DNA chips is committed. The result of the selection is transmitted. A date until when a customer submits the sample is entered.

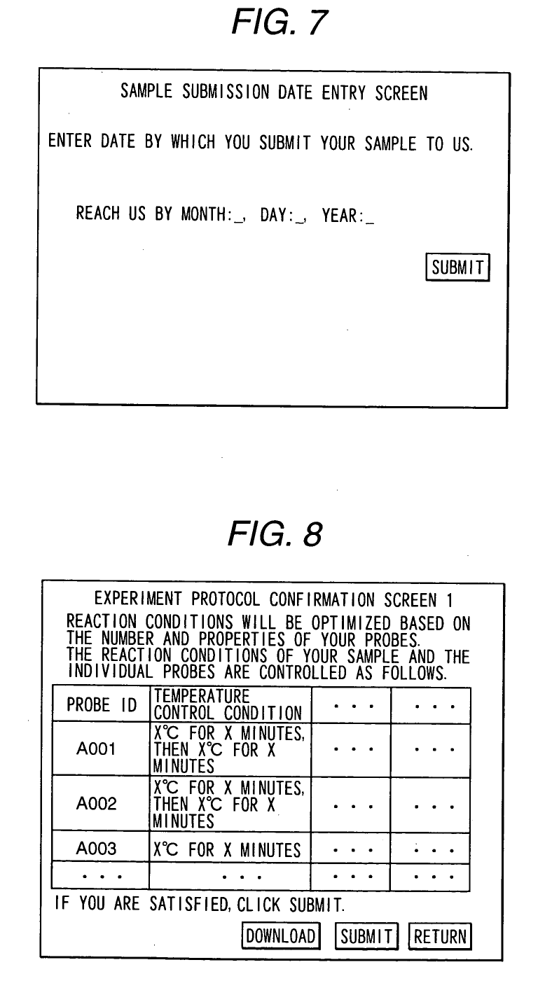

The screen shown in FIG. 7 is a screen presented in this processing (screen for entering a date of shipping a sample).

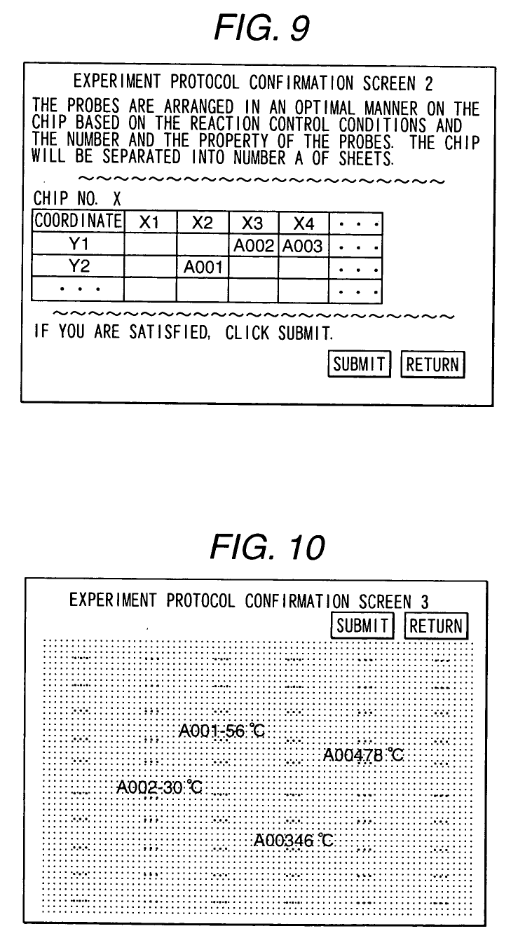

Experiment protocol is confirmed (Sample Submission Date Entry Screen) (S7). FIG. 8, FIG. 9, and FIG. 10 are screens shown in this processing. FIG. 8 shows Experiment Protocol Confirmation Screen 1, the screen is used for optimizing reaction conditions (setting the reaction conditions), and control (control conditions) methods for the reaction conditions for the sample and the individual probes are set as in the figure. As a typical control condition, a temperature control condition is set for the individual probes as in the figure.

With this, orders for DNA chip production from customers are accepted, an arrangement of individual nucleic acid probes are optimized for the genetic analysis using DNA chips at least requiring the temperature control according to Tm values of the nucleic acid probes, and a dispersion of temperature in a chip, the number of steps for the temperature control, and control periods are set.

FIG. 9 shows Experiment Protocol Confirmation Screen 2, the screen is used for arrangement setting for optimizing the arrangement of the probes in the individual areas on the chip based on the reaction control conditions and the number and the properties of the probes. Also how many sheets of the chips are divided into, for example number “A” of sheets is displayed. This screen indicates the individual areas in X coordinate and Y coordinate, and shows the probe arrangement.

FIG. 10 shows Experiment Protocol Confirmation Screen 3, and the screen shows the temperature conditions for the specified individual probe arrangement, and is used for confirmation.

The experiment protocol is downloaded as needed (S8).

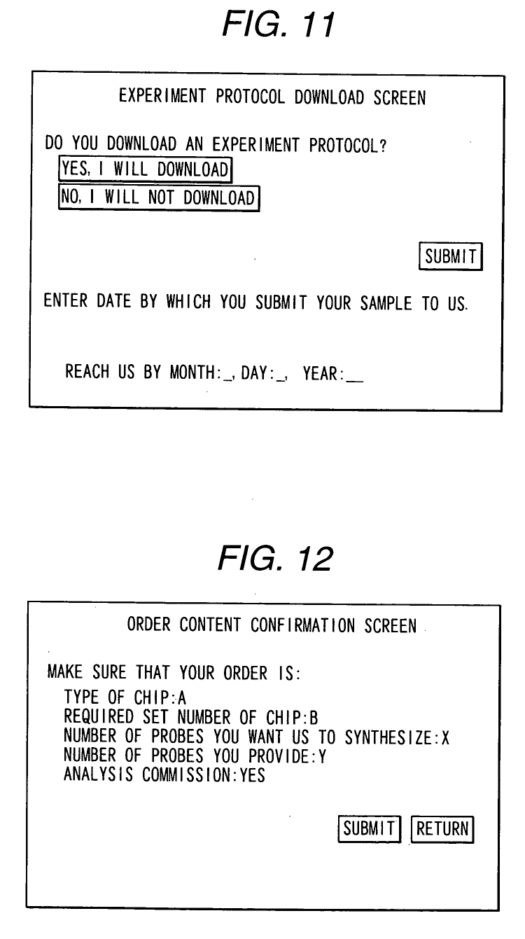

The screen shown in FIG. 11 is a screen presented in this processing (Experiment Protocol Download Screen), and the download is entered if necessary.

The content of the order is confirmed (S9).

The screen shown in FIG. 12 is a screen presented in this processing (Order Content Confirm Screen). The type of chips, the required number of sets of chip, the number of probes requested to synthesize by commission, the number of probes the customer provides, and the existence of analysis by commission are confirmed on this screen. The price or the price and the delivery date is confirmed (S10).

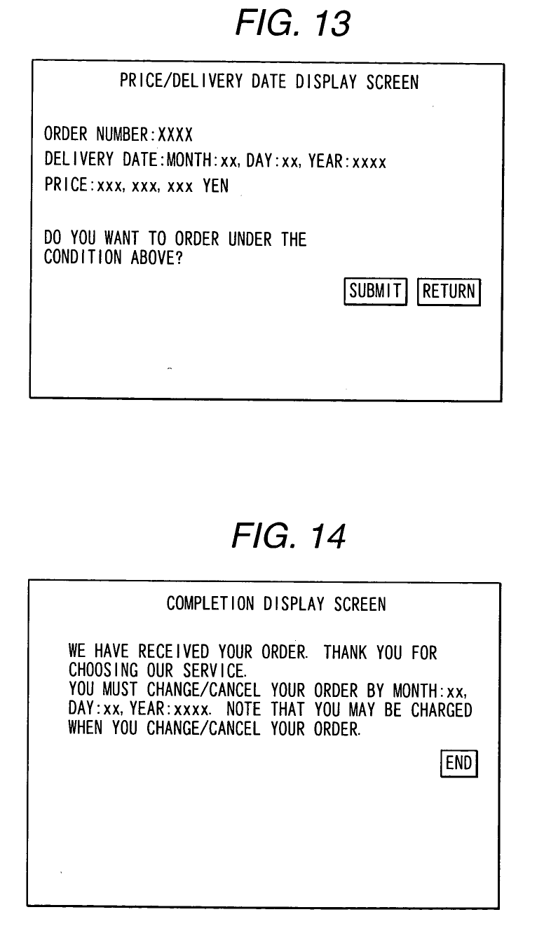

The screen shown in FIG. 13 is a screen presented in this processing (Price/Delivery Date Display Screen), which shows the number of orders, the delivery date, and the price, asks if to place the order. The result is entered.

With this process, the order for the production and the analysis is accepted (S11). In this case, when an order for the production and the analysis indicated in S11 is [accepteded] accepted in addition to the order for the production indicated in S4, the step of S4 may be bypassed.

A final confirmation is made (S12). The screen shown in FIG. 14 is a screen presented in this processing (Completion Display Screen), and shows the completion display.

As described above, a device is constituted for [acceoting] accepted orders for producing DNA chip from customers, determining an arrangement of the probes in a chip described in the order information file, and a dispersion of temperature in chips, registering the arrangement and the control conditions for the individual probe positions in the probe information file and an image information file for genetic analysis using DNA chips at least requiring the temperature control according to Tm values of the nucleic acid probes, and showing a screen by mapping a figure indicating the probe arrangement and the control conditions on the individual probe positions on an image indicating the chip from the latest image information file.

As indicated in FIG. 1, there is the DNA chip order accepting system 1 as a center of this system as described before, and other systems 25 (such as the production control system and the inventory control system), the order placing system for customer 70 (customer terminal), and the like are connected.

The DNA chip order receiving system calculates the price required for producing and analyzing DNA chips, and the delivery date based on the inventory control system, the customer system and the like, and notify the customer of them. A DNA chip order receiving center controls different types of information (overall information on DNA chips, information on production cost, production control information, inventory information, customer order information, and optimization parameter information for experiment conditions) entered by a system administrator, a production administrator, and an inventory administrator.

The production control system sends information relating to the production and the schedule of the DNA chip to the DNA chip order receiving system periodically or at any time. The inventory control system sends information on materials required for producing DNA chips to the DNA chip order receiving system periodically or at any time.

Individual customers notify the DNA chip order receiving system from individual systems through communication lines.

The order receiving system administrator enters information on DNA chips, information on optimization of experiment conditions (at least temperature conditions) using types of DNA chips that are ordered, and information on the arrangement of the nucleic acid probes as required to make addition and modification.

The following section describes the DNA chip order receiving center in detail.

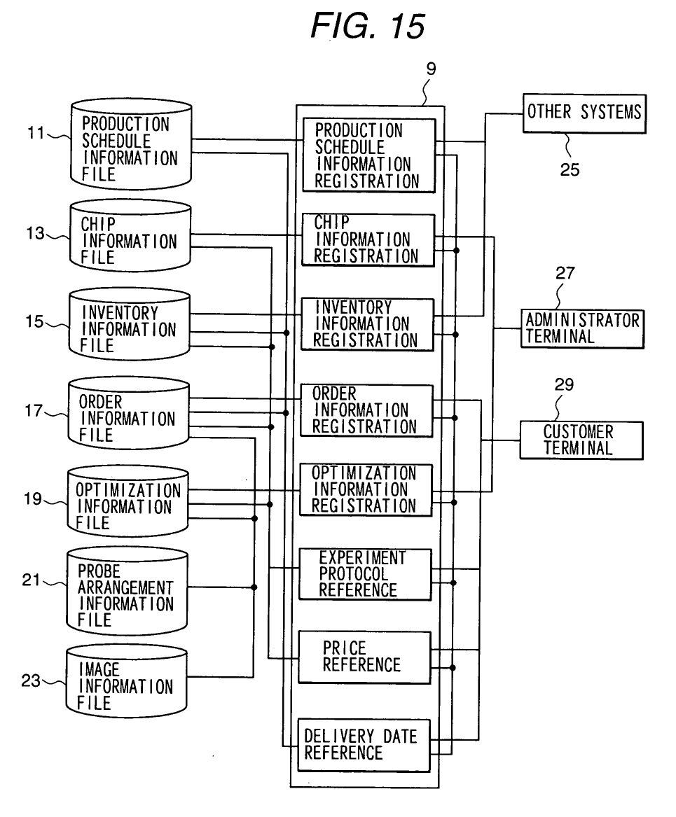

FIG. 15 shows processing features and file processing offered by the DNA chip order receiving center.

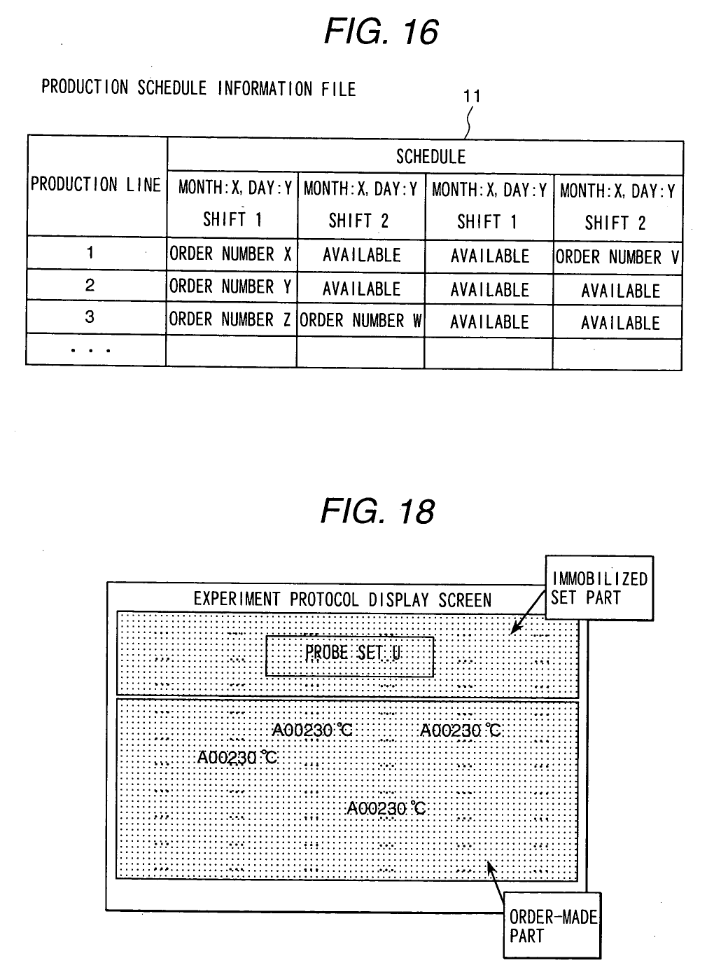

FIG. 16 to FIG. 22 show contents of individual files.

In FIG. 15, the DNA chip order receiving center 9 includes a production schedule information file 11, a DNA chip information file 13, an inventory information file 15, an order information file 17, an optimization information file 19, a probe arrangement information file 21, and an image information file 23.

The production schedule information file 11 records schedules for individual production lines and the like as shown in FIG. 16.

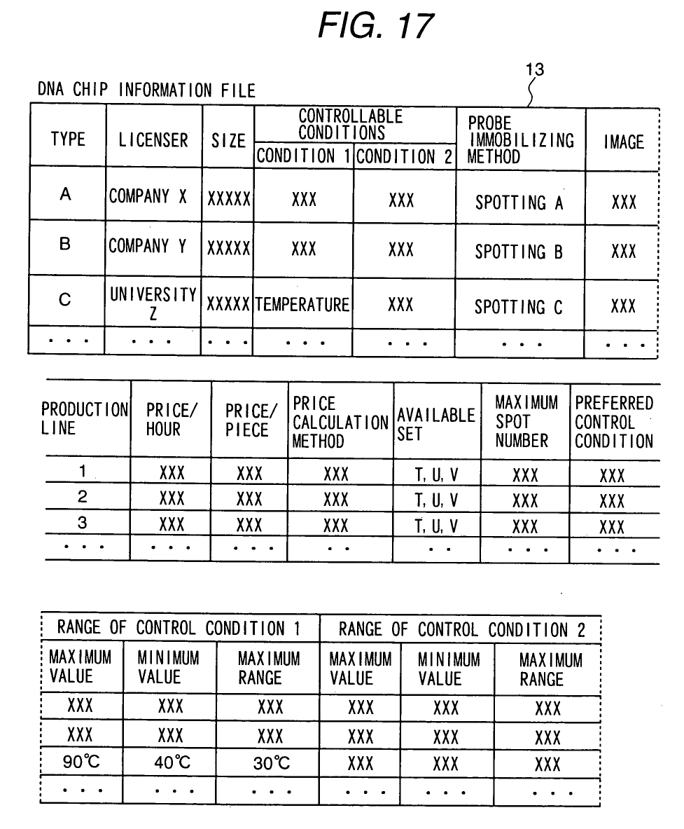

The DNA chip information file 13 records licensers of the individual types of chips, conditions to be controlled, methods for immobilizing probes, image data, lines used for production, prices per hour, cost and price per sheet of chips, methods for calculating the price, sets of available ready-made probes and the like as shown in FIG. 17. As the set of available ready-made probes, a set of cDNA pieces derived from human genes relating to cancer, a set of cDNA pieces derived from human genes relating to apoptosis, and A phage DNA pieces are possible.

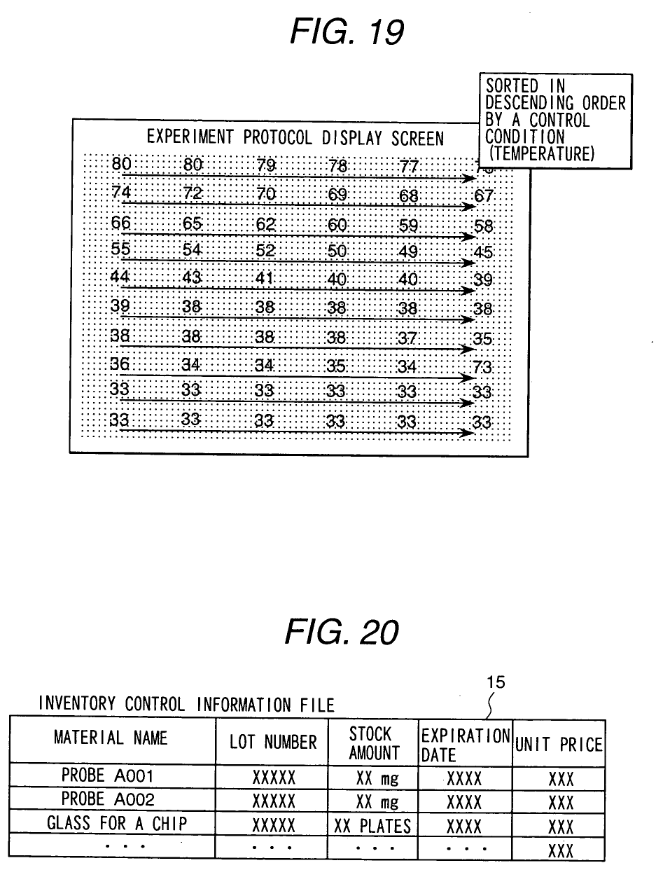

The DNA chip information file is produced by arranging the probes in a descending order with respect to a certain control condition (such as temperature), or produced additionally providing the individual probes with another condition (such as voltage). FIG. 19 shows rows of diffusion probes on a DNA chip produced by arranging the probes with temperature as a preferred condition. As shown in FIG. 19, individual areas presented on the Experiment Protocol Display Screen are arranged in descending order with respect to a control condition, temperature in this case.

[Fig.] FIG. 18 shows an example of a DNA chip where one [used such that a] part [of it is] ready-made and the [rest] remaining part [of it] is left [as ordermade] to be made according to an order. As shown in FIG. 18, the individual area shown on the Experiment Protocol Display Screen comprises [an] a fixed set part (readymade part) on the upper side[,] and an order-made part on the lower side.

The inventory control information file 15 records lot numbers for materials used for producing DNA chips, stock amounts, expiration dates, unit prices, and the like as shown in FIG. 20.

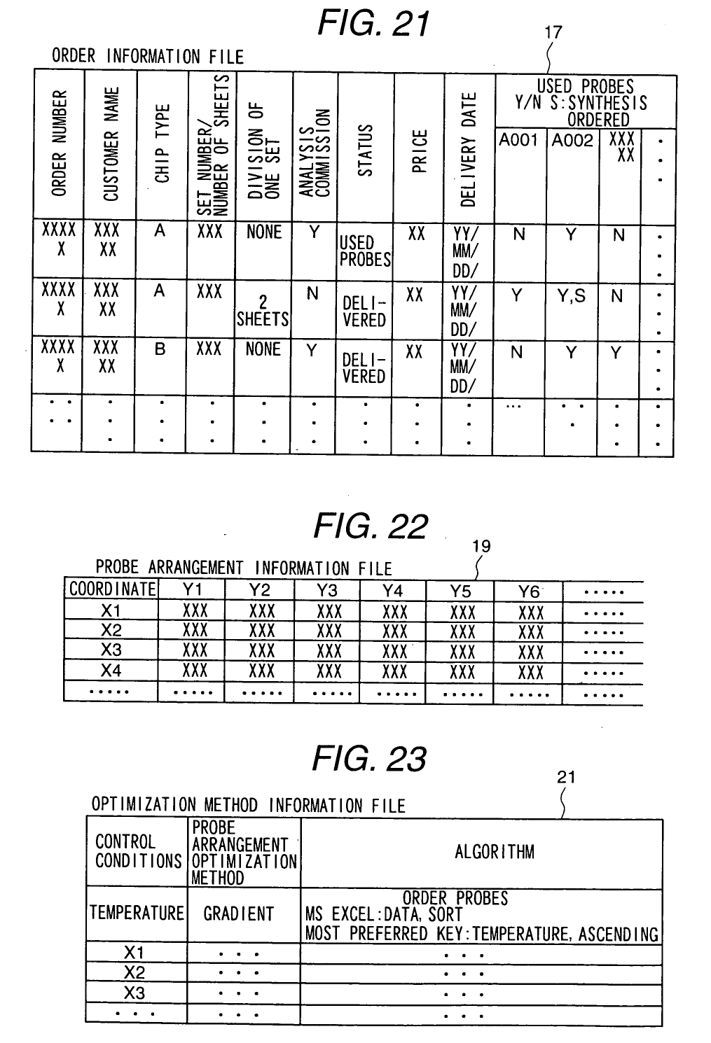

The order information file 17 records customer names, chip types, number of sets, number of sheets per set (number of probes, which are divided into two or more sheets according to the optimization condition), [existences] indication of whether analysis [by commission] is commissioned, order [statuses] status, prices, delivery dates, and probes to be used as shown in FIG. 21.

The probe arrangement information file 21 records probe names arranged at individual coordinate positions on a chip and the like as shown in FIG. 22.

The optimization information file 19 records information on optimization methods (arrangement methods) for the probe arrangement under the individual control conditions as shown in FIG. 23.

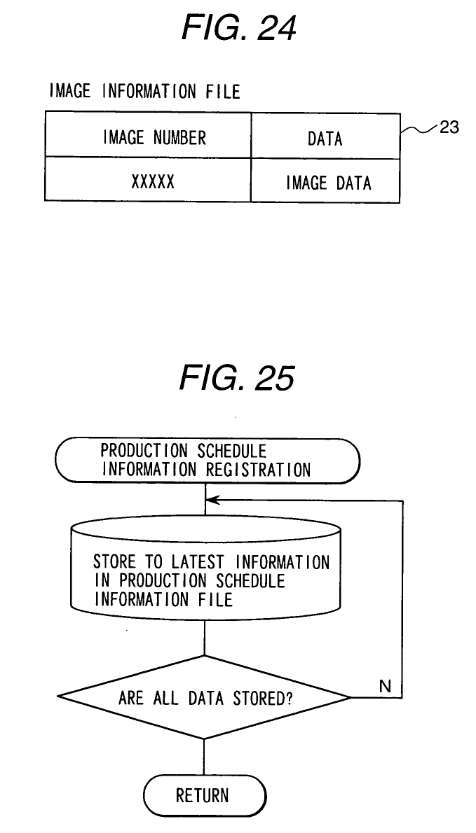

The image information file 23 records image numbers, image data, and the like as shown in FIG. 24.

The DNA chip order receiving system includes production schedule registration, chip information registration, inventory information registration, order information registration, optimizing information registration, experiment protocol reference, price reference, and delivery date reference.

The production schedule registration includes a process that receives different types of information relating to a production schedule sent from the production control system, and registers it to relating files (such as the production schedule information file) as the latest information, and a process that is invoked from a terminal provided for the system and registers different information relating to the production schedule to the production schedule information file as the latest information.

The chip information registration includes a process that receives different types of information relating to the DNA chip sent from the administrator of the DNA chip order receiving system, and registers it to relating files (such as the chip information file) as the latest information, and a process that is invoked from a terminal provided for the system and registers different information relating to the DNA chip to the chip information file as the latest information.

The inventory information registration includes a process that receives different types of information relating to the inventory control sent from the inventory control system, and registers it to relating files (such as the inventory information file) as the latest information, and a process that is invoked from a terminal provided for the system and registers different information relating to the production schedule to the inventory information file as the latest information.

The order information registration includes a process that receives different types of information relating to an order sent from a customer, and registers it to relating files (such as the order information file) as the latest information, and a process that is invoked from a terminal provided for the system and registers different information relating to an order into the order information file as the latest information.

The optimization information registration includes a process that receives different types of information relating to the optimization for probe arrangement used for a DNA chip sent from the administrator of the DNA chip order receiving system, and registers it to relating files (such as the optimization information file) as the latest information, and a process that is invoked from a terminal provided for the system and registers different information relating to the optimization for arrangement of probes produced by commission to the optimization information file as the latest information.

The experiment protocol reference includes a process that reads in the order information file and the optimization information file, determines an arrangement of the probes in a chip described in the order information file according to the methods described in the optimization information file, and [register] register the arrangement and the control conditions for individual probe positions to the probe information file and the image information file, and executes a process that maps a figure indicating the probe arrangement and the control conditions for the individual probe positions on an image indicating the chip from the latest image information file for showing it on a customer terminal.

As a method for determining an optimal probe arrangement, for example, there is a method where the information on the individual probes is sorted with the Tm value of the probes as key, and the probes are assigned in the ascending or descending order to coordinate numbers starting from the smallest value on a chip[,].

The price reference includes a process that reads in the chip information file, the inventory information file, the order information file, and the optimization information file, calculates a price, and shows it on a customer terminal, and the process that updates the order information file with the price.

As a method for determining the price, for example, there is a method [where assigning] which assigns a higher price on an experiment with more difficult control conditions.

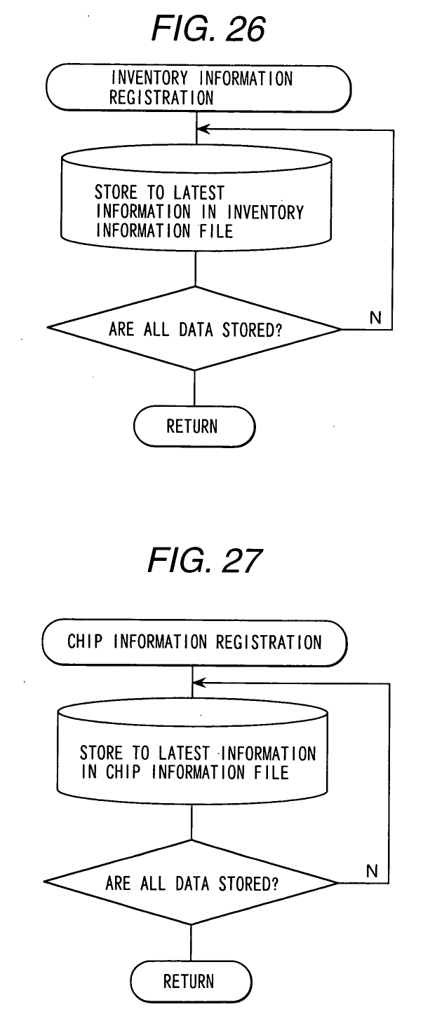

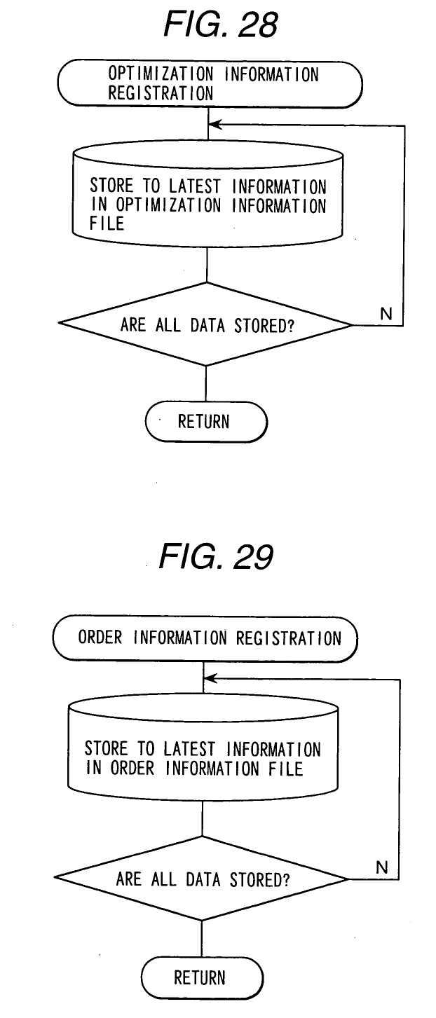

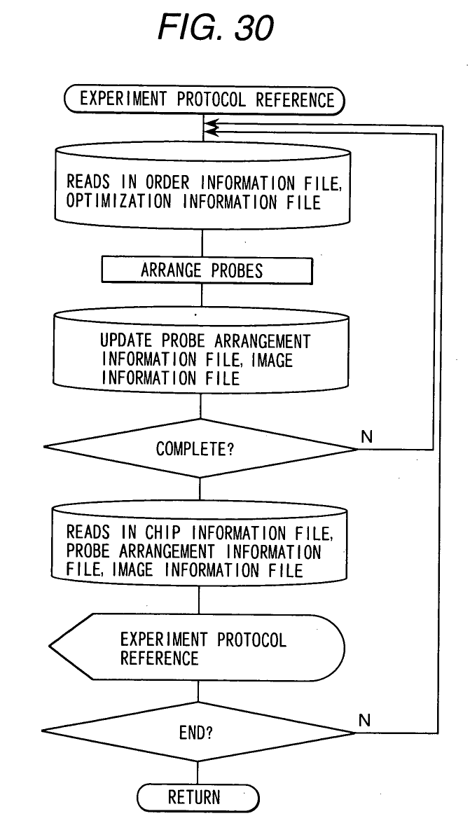

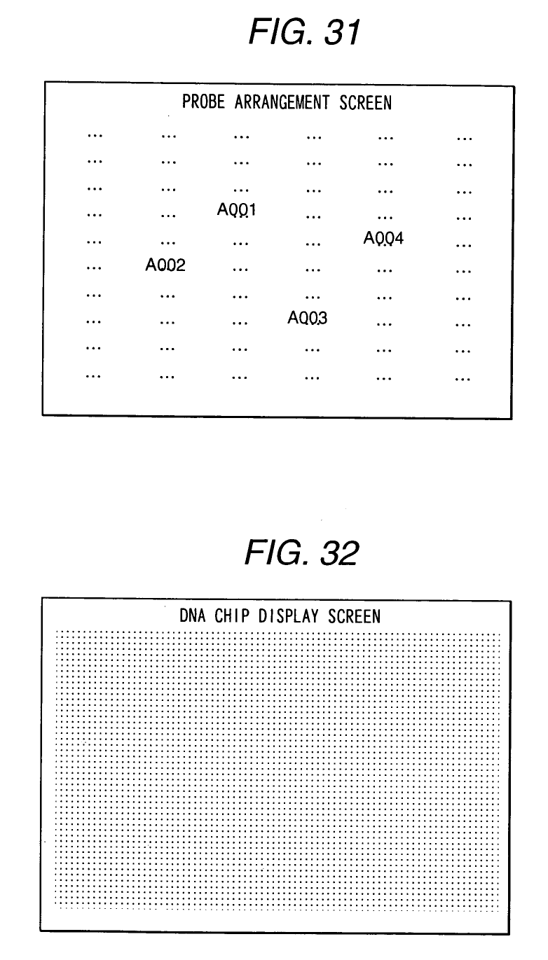

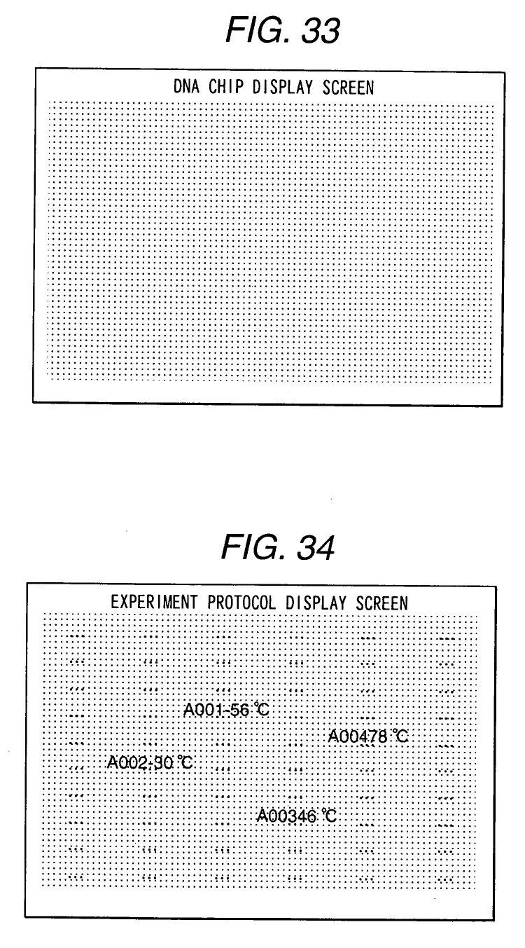

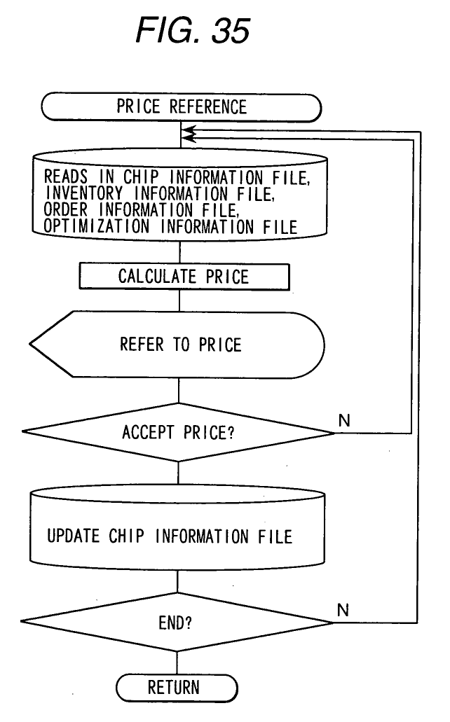

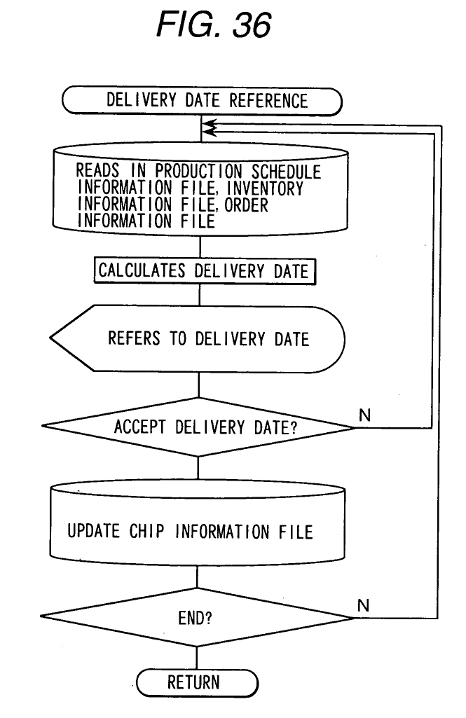

The delivery date reference includes a process that reads in the production schedule information file, the inventory information file, and the order information file, calculates the delivery date, and shows it on a customer terminal, and a process that updates the order information file with the delivery date.

[Fig.] FIG. 25 shows a flow of the production schedule information registration. In this process, the production schedule information is entered through the production control system, and the production schedule information file is updated with the information.

[Fig.] FIG. 26 shows a flow of the inventory information registration. In this process, the inventory information is entered through the inventory control system, and the inventory information file is updated with the information.

[Fig.] FIG. 27 shows a flow of the chip information registration. In this process, the chip information is obtained, and the information on chips is updated with the information.

[Fig.] FIG. 28 shows a flow of the optimization information registration. In this process, the system administrator enters information relating to the experiment optimization conditions, and the optimization file is updated with the information.

[Fig.] FIG. 29 shows a flow of the order information registration. In this process, the customer enters an order, and the order information file is updated with the information.

[Fig.] FIG. 30 shows a flow of the experiment protocol reference. In this process, the order information file and the optimization information file are read in, and the arrangement of the probes is optimized. The probe information arrangement information file and the image information file are updated with the arrangement.

[Fig.] FIG. 31 is an example of the image information showing an arrangement of the probes, and FIG. 32 is an example of an image showing control conditions for the individual probe positions. Further, the latest information is mapped from the image information file to the image information for the DNA chip.

[Fig.] FIG. 33 is an example of image information of a DNA chip, and FIG. 34 is an example of the image display for the information[,].

[Fig.] FIG. 35 shows a flow of the price reference. In this process, the chip information file, the inventory information file, the order information file, and the optimization information file are read in, a price for production or a price for order reception and a price for degree of difficulty of the experiment control are calculated, and the prices are displayed. After a customer confirms them, the order information file is updated with the prices.

[Fig.] FIG. 36 shows a flow of the delivery date reference. In this process, the production schedule information file, the inventory information file, and the order information file are read in, and the delivery date is calculated from the production schedule and the like, and is displayed. After a customer confirms it, the order information file is updated with the delivery date.

With the embodiment described above, an order for a DNA chip is accepted efficiently and quickly, and a customer is informed of the price and the delivery date. A researcher studying in the [filed] field of [gene with] genetics using DNA chips can increase the efficiency of their research by using the system. In some cases, a dealer of DNA chips may commit chip production] produce commissioned chips.

The constitution described above provides an order accepting method for producing DNA chips where a DNA chip is produced by immobilizing nucleic acid probes having specific base sequences in individual independent areas on a surface of a substrate, the type and the set number of the DNA chip are received as an order through a communication network, an arrangement of the nucleic acid probes in the individual areas is set, temperatures in the individual areas and temperature control conditions are shown on a screen, the screen is sent through the communication network for confirmation, and the production and analysis is ordered based on the content of the confirmed screen display through the communication network.

An order accepting method for producing DNA chips where the arrangement of the nucleic acid probes in individual areas is set based on a tendency of reducing dispersion of temperatures in the individual areas is provided.

An order accepting method for producing DNA chips where the experiment protocol display screen is constituted by composing an experiment control condition display screen with an nucleic acid probe arrangement screen is provided.

An order accepting method for producing DNA chips where the price is calculated while referring to a probe information file, an inventory information file, an order information file, and a file for setting information on the nucleic acid probes is provided.

An order accepting system for producing DNA chips that immobilizes nucleic acid probes having specific base sequences in individual independent areas on a surface of a substrate to produce DNA chips, and comprises an order receiving device for receiving the type and the set number of a DNA chip through a communication network, an arrangement setting device for setting the arrangement of the nucleic acid probes in the individual areas, a screen device for displaying the probe arrangement and the probe properties in the individual areas, and a production order accepting device for sending confirmation information using a screen through the communication network, and accepting an order for production based on the content of a confirmed screen display through the communication network is provided.

An order accepting system for producing DNA chips that immobilizes nucleic acid probes having specific base sequences in individual independent areas on a surface of a substrate to produce DNA chips, and comprises an order receiving device for receiving the type and the set number of a DNA chip through a communication network, an arrangement setting device for setting the arrangement of the nucleic acid probes in the individual areas, a screen device for displaying the probe arrangement and control conditions for the individual nucleic acid probe positions, and a production/analysis order accepting device for sending confirmation information using a display screen through the communication network, and accepting an order for production and analysis based on the content of a confirmed screen display through the communication network is provided.

An order accepting system for producing DNA chips that is required to control temperature according to the melting temperature (Tm) value of nucleic acid probes, and comprises an arrangement setting device for setting an arrangement of the nucleic acid probes recorded on an order information file in individual areas in a chip, a register processing device for registering the arrangement of the nucleic acid probes in the individual areas, and control conditions for individual probe positions to an image information file, an image display device for mapping a drawing indicating the probe arrangement and the control conditions at the individual probe positions on the individual areas on a screen indicating a DNA chip, and a production/analysis order accepting device for sending confirmation information using a display screen through a communication network, and accepting an order for production and analysis based on the content of a confirmed screen display through the communication network is provided.

An order accepting system for producing DNA chips provided with a price calculation processing device for calculating prices while referring to a probe information file for recording an arrangement of nucleic acid probes in individual areas, and control conditions for individual probe positions, an inventory information file, and an order information file is provided.

An order accepting system for producing DNA chips that is provided with a delivery date calculating device, which compares a content of an order, an inventory status and a production schedule with one another to calculate the delivery date, and displays a calculated delivery date on a screen display device is provided.

The present embodiment is an example of the present invention, and it is not intended to limit the extent of the present invention to this embodiment. For example, there are many variations in the experiment control conditions used for optimizing the probe arrangement, the method for optimization under these conditions, the method for calculating the price, and the method for calculating the delivery date.

1. A method of accepting commission production of DNA chips, wherein nucleic acid probes having specific base sequences are immobilized in independent areas on a surface of a substrate thereby to produce the DNA chips, which comprises the steps of:
receiving the type and the set number of a DNA chip through a communication network;
setting an arrangement of nucleic acid probes in individual areas;
displaying a drawing for showing the nucleic acid probes and probe property data at individual nucleic acid probe positions on a screen;
sending the display screen through the communication network for confirmation; and
accepting an order for production based on a confirmed screen display through the communication network.

2. A method of accepting commission production of DNA chips, wherein nucleic acid probes having specific base sequences are immobilized in independent areas on a surface of a substrate thereby to produce the DNA chips, which comprises the steps of:
receiving the type and the set number of a DNA chip through a communication network;
setting an arrangement of nucleic acid probes in individual areas;
displaying a drawing for showing the nucleic acid probes and probe property data at individual nucleic acid probe positions on a screen;
sending the display screen through the communication network for confirmation;
providing price information from a memory device for storing the price information; and
accepting an order for production based on a confirmed screen display through the communication network.

3. A method of accepting commission production of DNA chips, wherein nucleic acid probes having specific base sequences are immobilized in independent areas on a surface of a substrate thereby to produce the DNA chips, which comprises the steps of:
receiving the type and the set number of a DNA chip through a communication network;
setting an arrangement of nucleic acid probes in individual areas;
displaying a drawing for showing the nucleic acid probes and probe property data at individual nucleic acid probe positions on a screen;
sending the display screen through the communication network for confirmation;
accepting an order for production based on a confirmed screen display through the communication network;
calculating the delivery date while comparing content of the order, an inventory status, and a production schedule with one another; and
providing the delivery information through the communication network.

4. A method of accepting commission production of DNA chips, wherein nucleic acid probes having specific base sequences are immobilized in independent areas on a surface of a substrate thereby to produce the DNA chips, which comprises the steps of:
receiving the type and the set number of a DNA chip through a communication network;
setting an arrangement of nucleic acid probes in individual areas;
displaying a drawing for showing the nucleic acid probes and control conditions at individual nucleic acid probe positions on a screen;
sending the display screen through the communication network for confirmation; and
accepting an order for production and analysis based on a confirmed screen display through the communication network.

5. A method of accepting commission production of DNA chips, wherein nucleic acid probes having specific base sequences are immobilized in independent areas on a surface of a substrate thereby to produce the DNA chips, which comprises the steps of:
receiving the type and the set number of a DNA chip through a communication network;
setting an arrangement of nucleic acid probes in individual areas;
displaying temperatures of the individual areas, and temperature control conditions on a screen;
sending the display screen through the communication network for confirmation; and
accepting an order for production and analysis based on a confirmed screen display through the communication network.

6. A method of accepting commission production of DNA chips, wherein nucleic acid probes having specific base sequences are immobilized in independent areas on a surface of a substrate thereby to produce the DNA chips, which comprises the steps of:
receiving the type and the set number of a DNA chip through a communication network;
setting an arrangement of nucleic acid probes in individual areas;
displaying temperatures of the individual areas, and temperature control conditions on a screen;
sending the display screen through the communication network for confirmation;
accepting an order for production and analysis based on a confirmed screen display through the communication network; and
setting the arrangement of the nucleic acid probes in the individual areas based on a tendency of reducing dispersion of the temperatures in the individual areas.

7. A method of accepting commission production of DNA chips, wherein nucleic acid probes having specific base sequences are immobilized in independent areas on a surface of a substrate thereby to produce the DNA chips, which comprises the steps of:
receiving the type and the set number of a DNA chip through a communication network;
setting an arrangement of nucleic acid probes in individual areas;
displaying temperatures of the individual areas, and temperature control conditions on a screen;
sending the display screen through the communication network for confirmation;
accepting an order for production and analysis based on a confirmed screen display through the communication network; and
constituting an experiment protocol display screen by composing a nucleic acid probe arrangement screen with an experiment control condition display screen.

8. A method of accepting commission production of DNA chips, wherein nucleic acid probes having specific base sequences are immobilized in independent areas on a surface of a substrate thereby to produce the DNA chips, which comprises the steps of:
receiving the type and the set number of a DNA chip through a communication network;
setting an arrangement of nucleic acid probes in individual areas;
displaying temperatures of the individual areas, and temperature control conditions on a screen;
sending the display screen through the communication network for confirmation;
accepting an order for production and analysis based on a confirmed screen display through the communication network; and
calculating a price while referring to a probe information file, an inventory information file, an order information file, and a setting information file for nucleic acid probe arrangement.

9. A method of accepting commission production of DNA chips, wherein nucleic acid probes having specific base sequences are immobilized in independent areas on a surface of a substrate thereby to produce the DNA chips, which comprises the steps of:
an order input device for receiving the type and the set number of a DNA chip through a communication network;
an arrangement setting device for setting an arrangement of nucleic acid probes in individual areas;
a screen device for displaying the probe arrangement and probe properties in the individual areas;
a device for sending the display screen through the communication network for confirmation; and
a device for displaying a part of or the all of the content of a received order.

10. A system of accepting commission production of DNA chips, wherein an order relating to production of DNA chips is received from customers, and it is required to control temperature according to melting temperature (Tm) value of the nucleic acid probes comprising:
an arrangement setting device for setting an arrangement of nucleic acid probes recorded in an order information file in individual areas on a chip;
a registration processing device for registering the arrangement of the nucleic acid probes in the individual areas, and control conditions at individual probe positions to an image information file;
a screen display device mapping a drawing for indicating the probe arrangement and the control conditions at the individual probe positions from the image information file on individual areas on a screen for indicating a DNA chip; and
a production/analysis order accepting device for sending confirmation information using the display screen through a communication network, and accepting an order for production and analysis based on a confirmed screen display through the communication network.

11. A system of accepting commission production of DNA chips, wherein an order relating to production of DNA chips from customers, and it is required to control temperature according to melting temperature (Tm) value of the nucleic acid probes:
an arrangement setting device for setting an arrangement of nucleic acid probes recorded in an order information file in individual areas on a chip;
a registration processing device for registering the arrangement of the nucleic acid probes in the individual areas, and control conditions at individual probe positions to an image information file;
a screen display device mapping a drawing for indicating the probe arrangement and the control conditions at the individual probe positions from the image information file on individual areas on a screen for indicating a DNA chip;
a production/analysis order accepting device for sending confirmation information using the display screen through a communication network, and accepting an order for production and analysis based on a confirmed screen display through the communication network; and
a price calculation processing device for calculating a price while referring to a probe information file for storing the arrangement of the nucleic acid probes in individual areas and the control conditions at individual probe positions, an inventory information file, and the order information file.

12. A system of accepting commission production of DNA chips, wherein the nucleic acid probes including specific base sequences are immobilized in independent areas on a surface of a substrate to produce the DNA chips or an order relating to production of DNA chips from customers, and it is required to control temperature according to melting temperature (Tm) value of the nucleic acid probes, and the calculated delivery date is displayed on a screen display device:
a delivery date calculation processing device for calculating a delivery date by comparing the content of an order, a inventory status, and a production schedule with one another.
